(19) United States
(12) Patent Application Publication
(10) Pub. No.: US 2019/0374202 A1
(43) Pub. Date: Dec. 12, 2019

(54) ULTRASOUND PROBE

(71) Applicant: QISDA CORPORATION, Taoyuan City (TW)

(72) Inventor: Chu-Chiang Chen, Taoyuan City (TW)

(21) Appl. No.: 16/409,847

(22) Filed: May 12, 2019

(30) Foreign Application Priority Data
Jun. 7, 2018 (TW) 107119631

(51) Int. Cl. A61B 8/00

(52) U.S. Cl. CPC A61B 8/4444; A61B 8/4281

(57) ABSTRACT

An ultrasound probe includes a casing, a multi-axis rotating mechanism, and an ultrasound imaging and treating module. The multi-axis rotating mechanism is disposed in the casing. The ultrasound imaging and treating module is disposed in the casing and connected to the multi-axis rotating mechanism. The multi-axis rotating mechanism drives the ultrasound imaging and treating module to rotate. The ultrasound imaging and treating module generates an ultrasound image and selectively treats a portion corresponding to the ultrasound image.

### BACKGROUND OF THE INVENTION

### 1. Field of the Invention

The invention relates to an ultrasound probe and, more particularly, to an ultrasound probe capable of performing diagnosis and treatment simultaneously when an ultrasound image is generated.

### 2. Description of the Prior Art

Since ultrasound scanning equipment does not destroy material structure and cell, the ultrasound scanning equipment is in widespread use for the field of material and clinical diagnosis. In general, a doctor may use an ultrasound probe to perform ultrasound scanning for a portion for inspection, so as to generate an ultrasound image of the portion. When the doctor determines that a nidus exists in the ultrasound image, the doctor has to use an individual treating tool to treat the nidus. At this time, the nidus may disappear from the ultrasound image due to vibration of the ultrasound probe, such that the doctor needs to perform ultrasound scanning for the portion again. In other words, since the doctor cannot perform diagnosis and treatment simultaneously when the ultrasound image is generated, medical efficiency is reduced.

### SUMMARY OF THE INVENTION

An objective of the invention is to provide an ultrasound probe capable of performing diagnosis and treatment simultaneously when an ultrasound image is generated, so as to solve the aforesaid problems.

According to an embodiment of the invention, an ultrasound probe comprises a casing, a multi-axis rotating mechanism, and an ultrasound imaging and treating module. The multi-axis rotating mechanism is disposed in the casing. The ultrasound imaging and treating module is disposed in the casing and connected to the multi-axis rotating mechanism. The multi-axis rotating mechanism drives the ultrasound imaging and treating module to rotate. The ultrasound imaging and treating module generates an ultrasound image and selectively treats a portion corresponding to the ultrasound image.

As mentioned in the above, the invention connects the ultrasound imaging and treating module to the multi-axis rotating mechanism and utilizes the multi-axis rotating mechanism to drive the ultrasound imaging and treating module to rotate, so as to perform ultrasound scanning for a portion for inspection and then generate an ultrasound image of the portion. When a doctor determines that a nidus exists in the ultrasound image, the doctor may immediately use the ultrasound imaging and treating module to treat the portion (i.e. nidus) corresponding to the ultrasound image. Accordingly, the invention can perform diagnosis and treatment simultaneously when an ultrasound image is generated, so as to enhance medical efficiency.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic view illustrating an ultrasound probe according to an embodiment of the invention.

FIG. 2 is a schematic view illustrating an ultrasound probe according to another embodiment of the invention.

FIG. 3 is a schematic view illustrating an ultrasound probe according to another embodiment of the invention.

FIG. 4 is a schematic view illustrating an ultrasound probe according to another embodiment of the invention.

### DETAILED DESCRIPTION

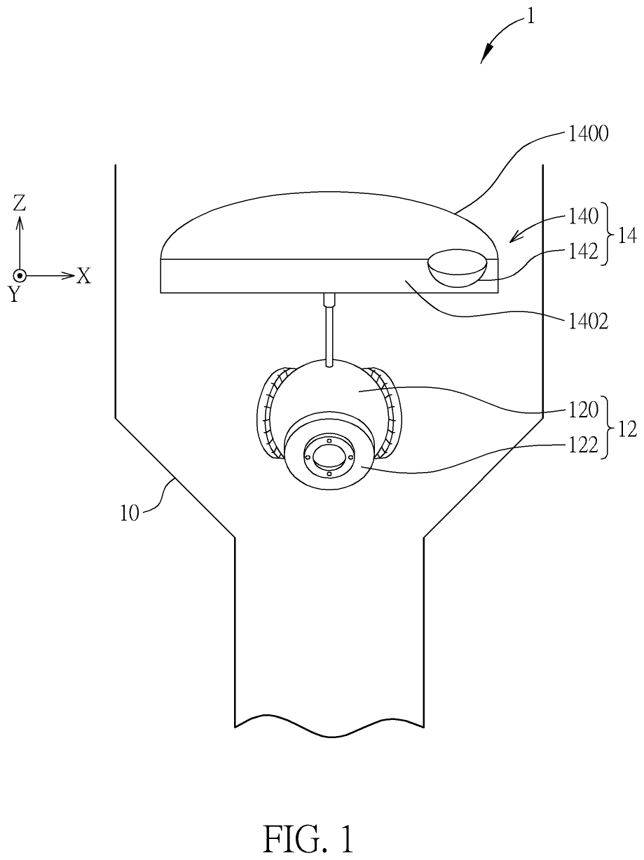

Referring to FIG. 1, FIG. 1 is a schematic view illustrating an ultrasound probe 1 according to an embodiment of the invention. As shown in FIG. 1, the ultrasound probe 1 comprises a casing 10, a multi-axis rotating mechanism 12, and an ultrasound imaging and treating module 14. The multi-axis rotating mechanism 12 is disposed in the casing 10. The ultrasound imaging and treating module 14 is disposed in the casing 10 and connected to the multi-axis rotating mechanism 12. The multi-axis rotating mechanism 12 is configured to drive the ultrasound imaging and treating module 14 to rotate. In general, the ultrasound probe 1 may be further equipped with some necessary hardware or software components for specific purposes, such as a controller, a circuit board, a memory, etc., and it depends on practical applications.

In this embodiment, the multi-axis rotating mechanism 12 comprises a spherical rotor 120 and a stator 122, wherein one of the spherical rotor 120 and the stator 122 may be fixed to the casing 10, and the ultrasound imaging and treating module 14 may be connected to another one of the spherical rotor 120 and the stator 122. As the embodiment shown in FIG. 1, the stator 122 is fixed to the casing 10 and the ultrasound imaging and treating module 14 is connected to the spherical rotor 120. Accordingly, when the spherical rotor 120 rotates with respect to the stator 122, the spherical rotor 120 drives the ultrasound imaging and treating module 14 to rotate. For further illustration, the spherical rotor 120 may rotate with respect to the stator 122 around X-axis, Y-axis, and/or Z-axis, so as to drive the ultrasound imaging and treating module 14 to rotate around X-axis, Y-axis, and/or Z-axis. It should be noted that the stator 122 may be fixed to the casing 10 by screw, welding, engagement, adhesion, rivet, or other fixing manners according to practical applications. In practical applications, an ultrasound conductive medium, a water bag, or a transparent protection cover (not shown) may be disposed on a front end of the casing 10, so as to cover the ultrasound imaging and treating module 14.

In this embodiment, the ultrasound imaging and treating module 14 comprises an ultrasound imaging unit 140 and a treating unit 142. As shown in FIG. 1, the ultrasound imaging unit 140 is connected to the spherical rotor 120 of the multi-axis rotating mechanism 12 and the treating unit 142 is connected to the ultrasound imaging unit 140. The ultrasound imaging unit 140 comprises an imaging region 1400 and a non-imaging region 1402. The ultrasound imaging unit 140 performs ultrasound scanning for a portion for inspection through the imaging region 1400, so as to generate an ultrasound image. Accordingly, the invention may connect the treating unit 142 to the non-imaging region 1402 (e.g. edge) of the ultrasound imaging unit 140, so as to prevent the imaging region 1400 of the ultrasound imaging unit 140 from being blocked by the treating unit 142.

When a doctor uses the ultrasound probe 1 to perform ultrasound scanning for a portion for inspection, the doctor may control the spherical rotor 120 of the multi-axis rotating mechanism 12 to rotate, so as to drive the ultrasound imaging and treating module 14 to rotate. At the same time, the ultrasound imaging unit 140 may perform ultrasound scanning for the portion through the imaging region 1400, so as to generate an ultrasound image. In this embodiment, an imaging range of the ultrasound imaging unit 140 covers a treating range of the treating unit 142. Therefore, when the doctor determines that a nidus exists in the ultrasound image, the doctor may immediately use the ultrasound imaging and treating module 14 to treat the portion (i.e. nidus) corresponding to the ultrasound image. In other words, the invention may use the ultrasound imaging and treating module 14 to generate an ultrasound image and use the ultrasound imaging and treating module 14 to selectively treat a portion corresponding to the ultrasound image when a nidus exists in the ultrasound image. Accordingly, the invention can perform diagnosis and treatment simultaneously when an ultrasound image is generated, so as to enhance medical efficiency. In practical applications, the ultrasound imaging unit 140 may be an ultrasound sensor and the treating unit 142 may be an ultrasound generating unit (e.g. high-intensity focused ultrasound (HIFU)), a puncture needle, or an electrosurgical unit, and it depends on practical applications.

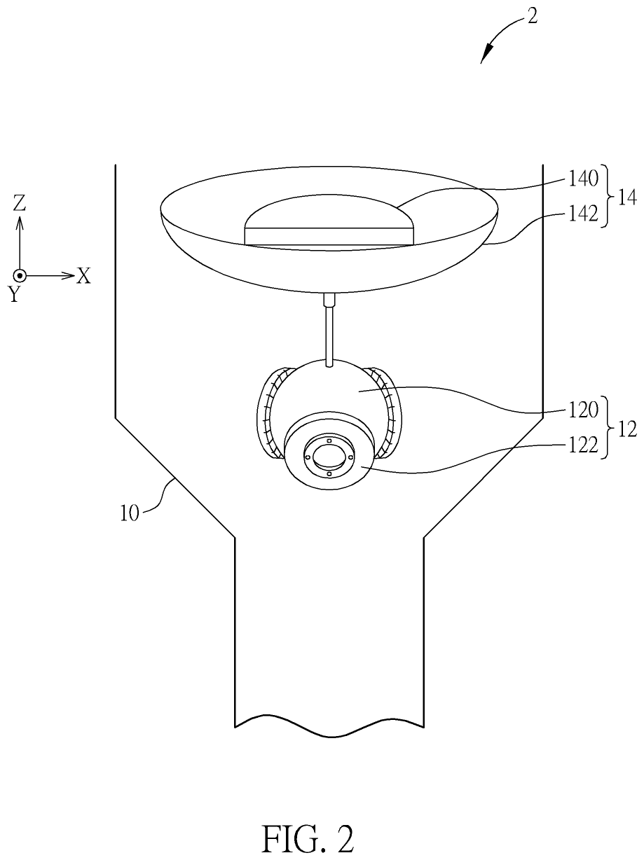

Referring to FIG. 2, FIG. 2 is a schematic view illustrating an ultrasound probe 2 according to another embodiment of the invention. The main difference between the ultrasound probe 2 and the aforesaid ultrasound probe 1 is that the ultrasound probe 2 connects the treating unit 142 of the ultrasound imaging and treating module 14 to the spherical rotor 120 of the multi-axis rotating mechanism 12 and connects the ultrasound imaging unit 140 of the ultrasound imaging and treating module 14 to the treating unit 142. Accordingly, when the spherical rotor 120 rotates with respect to the stator 122, the spherical rotor 120 drives the ultrasound imaging and treating module 14 to rotate.

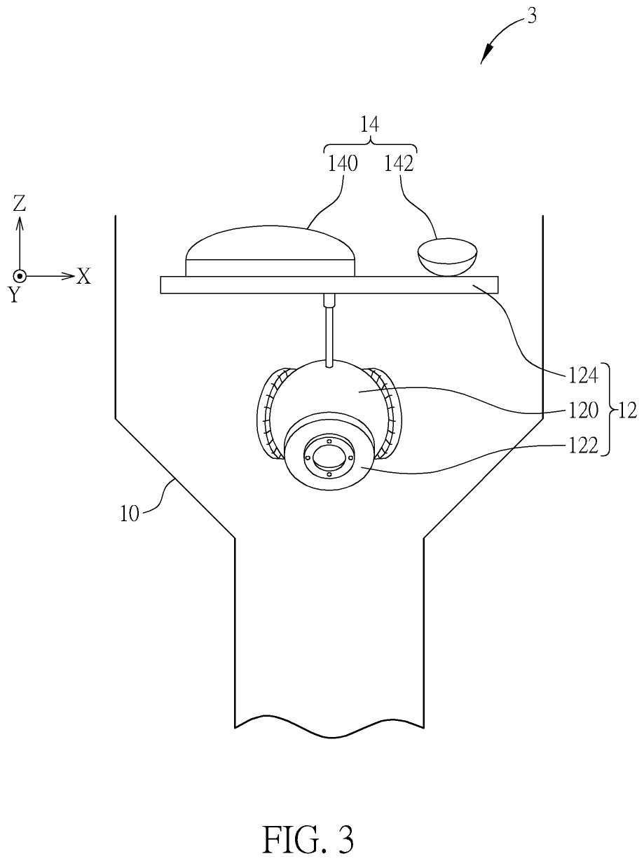

Referring to FIG. 3, FIG. 3 is a schematic view illustrating an ultrasound probe 3 according to another embodiment of the invention. The main difference between the ultrasound probe 3 and the aforesaid ultrasound probe 1 is that the multi-axis rotating mechanism 12 of the ultrasound probe 3 comprises a support base 124, as shown in FIG. 3. In this embodiment, the support base 124 may be connected to the spherical rotor 120, and the ultrasound imaging unit 140 and the treating unit 142 of the ultrasound imaging and treating module 14 are connected to the support base 124. Accordingly, when the spherical rotor 120 rotates with respect to the stator 122, the spherical rotor 120 drives the support base 124 and the ultrasound imaging and treating module 14 to rotate.

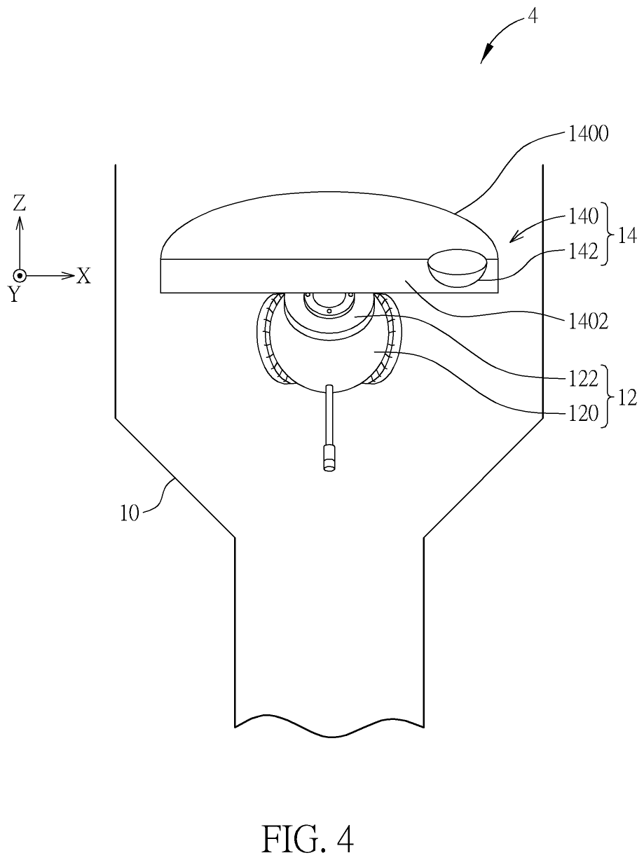

Referring to FIG. 4, FIG. 4 is a schematic view illustrating an ultrasound probe 4 according to another embodiment of the invention. The main difference between the ultrasound probe 4 and the aforesaid ultrasound probe 1 is that the ultrasound probe 4 fixes the spherical rotor 120 of the multi-axis rotating mechanism 12 to the casing 10, such that the stator 122 may rotate with respect to the spherical rotor 120. As shown in FIG. 4, the ultrasound imaging and treating module 14 is connected to the stator 122 of the multi-axis rotating mechanism 12. Accordingly, when the stator 122 rotates with respect to the spherical rotor 120, the stator 122 drives the ultrasound imaging and treating module 14 to rotate. It should be noted that the spherical rotor 120 may be fixed to the casing 10 by screw, welding, engagement, adhesion, rivet, or other fixing manners according to practical applications. Furthermore, the treating unit 142 shown in FIG. 2 or the support base 124 shown in FIG. 3 may also be connected to the stator 122 of the multi-axis rotating mechanism 12 shown in FIG. 4 according to practical applications.

As mentioned in the above, the invention connects the ultrasound imaging and treating module to the multi-axis rotating mechanism and utilizes the multi-axis rotating mechanism to drive the ultrasound imaging and treating module to rotate, so as to perform ultrasound scanning for a portion for inspection and then generate an ultrasound image of the portion. When a doctor determines that a nidus exists in the ultrasound image, the doctor may immediately use the ultrasound imaging and treating module to treat the portion (i.e. nidus) corresponding to the ultrasound image. Accordingly, the invention can perform diagnosis and treatment simultaneously when an ultrasound image is generated, so as to enhance medical efficiency.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An ultrasound probe comprising:
a casing;
a multi-axis rotating mechanism disposed in the casing; and
an ultrasound imaging and treating module disposed in the casing and connected to the multi-axis rotating mechanism, the multi-axis rotating mechanism driving the ultrasound imaging and treating module to rotate, the ultrasound imaging and treating module generating an ultrasound image and selectively treating a portion corresponding to the ultrasound image.

2. The ultrasound probe of claim 1, wherein the multi-axis rotating mechanism comprises a spherical rotor and a stator, one of the spherical rotor and the stator is fixed to the casing, and the ultrasound imaging and treating module is connected to another one of the spherical rotor and the stator.

3. The ultrasound probe of claim 1, wherein the ultrasound imaging and treating module comprises an ultrasound imaging unit and a treating unit, the ultrasound imaging unit is connected to the multi-axis rotating mechanism, and the treating unit is connected to a non-imaging region of the ultrasound imaging unit.

4. The ultrasound probe of claim 3, wherein an imaging range of the ultrasound imaging unit covers a treating range of the treating unit.

5. The ultrasound probe of claim 3, wherein the treating unit is an ultrasound generating unit, a puncture needle, or an electrosurgical unit.

6. The ultrasound probe of claim 1, wherein the ultrasound imaging and treating module comprises an ultrasound imaging unit and a treating unit, the treating unit is connected to the multi-axis rotating mechanism, and the ultrasound imaging unit is connected to the treating unit.

7. The ultrasound probe of claim 6, wherein an imaging range of the ultrasound imaging unit covers a treating range of the treating unit.

8. The ultrasound probe of claim 6, wherein the treating unit is an ultrasound generating unit, a puncture needle, or an electrosurgical unit.

9. The ultrasound probe of claim 1, wherein the ultrasound imaging and treating module comprises an ultrasound imaging unit and a treating unit, the multi-axis rotating mechanism comprises a support base, and the ultrasound imaging unit and the treating unit are connected to the support base.

10. The ultrasound probe of claim 9, wherein an imaging range of the ultrasound imaging unit covers a treating range of the treating unit.

11. The ultrasound probe of claim 9, wherein the treating unit is an ultrasound generating unit, a puncture needle, or an electrosurgical unit.
